(19) United States
(12) Patent Application Publication
(10) Pub. No.: US 2025/0387171 A1
(43) Pub. Date: Dec. 25, 2025

(54) AUTOMATICALLY CONTROLLING AN INTEGRATED INSTRUMENT

(71) Applicant: The Regents of the University of California, Oakland, CA (US)

(72) Inventors: Laura Marcu, Davis, CA (US); Julien Bec, Davis, CA (US); Orin Bloch, Sacramento, CA (US)

(73) Assignee: The Regents of the University of California, Oakland, CA (US)

(21) Appl. No.: 18/880,484

(22) PCT Filed: Aug. 29, 2023
(86) PCT No.: PCT/US2023/073050

§ 371 Date: Dec. 31, 2024

Related U.S. Application Data
(60) Provisional application No. 63/402,611, filed on Aug. 31, 2022.

(51) Int. Cl. A61B 34/20; A61B 34/00; A61B 34/10; A61B 90/00; A61B 90/20

(52) U.S. Cl. CPC A61B 34/20; A61B 34/10; A61B 34/25; A61B 90/20; A61B 90/37; A61B 2034/2055; A61B 2034/254; A61B 2090/3735

(57) ABSTRACT

A system, apparatus and method are provided for operating a medical instrument with an automated surgical system such that the instrument starts and stops automatically depending on its location, orientation, and/or other information. Thus, instead of requiring continual supervision by a surgeon and/or other personnel, one or more functions of the instrument can be activated, paused, reactivated, or deactivated through normal manipulation of the instrument without interrupting or impeding the work flow. A volume of operation for a medical procedure may be identified manually or automatically, based on the nature of the procedure, the part of a patient's body involved in the procedure, and/or other factors. When the automated surgical system detects entry of the instrument into the volume of operation, the instrument is automatically activated and is subsequently automatically deactivated upon departure from the volume of operation.

### RELATED APPLICATION(S)

This application claims the benefit of U.S. Provisional Patent Application No. 63/402,611, which was filed 31 Aug. 2022 and is incorporated by reference herein.

### GOVERNMENT LICENSE RIGHTS

This invention was made with U.S. Government support under Grant Nos. R01CA250512 and R01CA187427, awarded by the National Institutes of Health. The U.S. Government has certain rights in the invention.

### BACKGROUND

This disclosure relates to the fields of surgery and electronics. More particularly, a system, apparatus and methods are provided for integrating and operating a medical instrument with an automated surgical system.

A variety of medical and surgical instruments have been developed to help surgeons measure tissue and/or characterize tissue type (e.g., identify vessels, detect cancerous tissue) during surgery or other medical procedure. Currently, these instruments must be manually controlled for the duration of their operation. In particular, the surgeon, a nurse, or a technician must start and stop operation of the instrument (e.g., to collect data) manually using a controller (e.g., a switch, a foot pedal).

Unfortunately, however, manual control of an instrument can disrupt the surgery, particularly if the surgeon must operate the controller and the controller is not in proximity to the surgical field. If some other person controls the instrument, he or she must regularly communicate with the surgeon, and this communication may also interrupt the workflow.

### SUMMARY

In some embodiments, systems, apparatus, and methods are provided for automatically controlling operation of a medical instrument with an automated surgical system such as a surgical navigation system and/or a surgical robot. Automatic control may include starting the instrument (e.g., turning on its function) and pausing or stopping it. In these embodiments, the instrument's operation automatically starts and stops based on its location, orientation, and/or other status. In particular, the instrument may be activated automatically as it approaches or enters a volume of operation surrounding a focus of a medical procedure (e.g., a patient's brain, a tumor), and likewise automatically deactivated when it leaves the volume of operation.

In some embodiments, the instrument or some other component of the automated surgical system measures or estimates the amount, intensity, and/or duration of exposure of a patient's tissue to light (e.g., a laser) or other radiation, and the radiation may automatically cease when a threshold is achieved. Yet further, the instrument may also (or instead) be configured to deliver a medication, marker, or other substance, and may automatically do so when the system determines that the instrument is properly positioned.

### DESCRIPTION OF THE FIGURES

FIG. 1 is a block diagram depicting an automated surgical system with an integrated instrument configured for automatic operation, in accordance with some embodiments.

FIG. 2 depicts a volume of operation defined for a relatively large area or volume, according to some embodiments.

FIG. 3 depicts a volume of operation defined for a relatively small area or volume, according to some embodiments.

FIG. 4 depicts the illustration of the location of an instrument integrated with an automated surgical system, within a volume of operation, according to some embodiments.

FIG. 5 is a flow chart demonstrating a method of controlling operation of an instrument integrated with an automated surgical system, according to some embodiments.

### DETAILED DESCRIPTION

The following description is presented to enable any person skilled in the art to make and use the disclosed embodiments, and is provided in the context of one or more particular applications and their requirements. Various modifications to the disclosed embodiments will be readily apparent to those skilled in the art, and the general principles defined herein may be applied to other embodiments and applications without departing from the scope of those that are disclosed. Thus, the present invention or inventions are not intended to be limited to the embodiments shown, but rather are to be accorded the widest scope consistent with the disclosure.

Systems, apparatus, and methods are provided for operating a medical instrument as part of an automated surgical system, such that the instrument is started and/or stopped automatically based on the type and location of the instrument (e.g., the instrument's function(s), how close or how far it is to/from the target tissue). In these embodiments, an automated surgical system may be or comprise a surgical robot, a surgical navigation system, or any other system or machinery that provides automated or semi-automated support of surgeries and/or other medical procedures.

In some implementations, for example, the instrument is capable of freehand tissue measurement and/or characterization, and comprises a hand-held probe for collecting data (e.g., spectroscopic data, reflectance, depth-resolved measurement) at a location that is identified and monitored by the automated surgical system. More specifically, the instrument may be configured to determine whether tissue at or near an operative portion of the instrument is healthy, tumorous, inflamed, or necrotic. It may do so, for example, by measuring the fluorescence signature of endogenous fluorophores naturally present in healthy or diseased tissues, and/or the fluorescence of one or more molecular probes administered to a patient. These measurements may be performed over different spectral bands (e.g., a fluorophore's entire emission spectra) and/or include time-resolved measurements. An associated console receives data collected by the probe and may distribute it to the automated surgical system and/or other equipment coupled to the system.

In particular, the console is coupled to both the probe and the automated surgical system and can relay data between these entities, including the data collected by the probe and/or information from the automated surgical system that indicates a location and/or orientation of the probe or that commands the probe to start or stop operation. In these embodiments, the probe starts operating automatically when the instrument or the system determines that it is within a specified range of the tissue, and stops automatically when it is out of the specified range. In some implementations involving brain surgery, for example, data collection may begin when the probe is within 5 mm of the outer surface of the brain.

The flow of the surgery is facilitated because no one need manually operate a controller, safety is enhanced because the instrument operates only when appropriate, and collected data are certain to be relevant because they were collected when the probe was positioned beneficially. The collected data may include spectroscopic data, reflectance, depth-resolved measurements, and/or other optical data, parameters, or images. The data may be displayed in concert with (e.g., overlaid upon) imagery obtained via Positron Emission Tomography or PET, Magnetic Resonance Imaging or MRI, Computed Tomography or CT, and/or other technologies. Further, the data may be overlaid, in real time, upon a microscope operated as part of the current medical procedure.

In some embodiments, position information regarding the probe may also be used to control light emitted by the instrument. This may be advantageous to avoid overexposing tissue and/or provide other benefits. For example, potential hazards may be avoided through automatic cessation of the instrument's radiation.

In some embodiments, determining the position of the optical instrument's hand-held probe is enabled by mounting upon the probe a tracking array comprising infrared-reflecting spheres that are tracked by the automated surgical system. Other types of markings or configurations may be employed in other embodiments to help the automated surgical system identify a precise location of an integrated instrument. The optical probe's characteristics (e.g., position and orientation of the active area with respect to the tracking array) may be calibrated manually and/or with a dedicated calibration tool.

For example, the volume of operation of a surgical field in which a surgery is performed may be determined by defining control points of an upper boundary (for example, points on the surface of the head in the case of brain surgery). Alternatively, the volume of operation can be determined from preoperative imaging data available within the automated surgical system (for example, the volume corresponding to a targeted brain tumor, plus an additional buffer). A tissue volume of operation can be reduced during the procedure by accounting for tissue removal. For example, part of a volume located within a skull where a probe is located may be assumed to have been resected.

Two-way communication between the optical instrument and the automated surgical system is provided by physical and/or wireless links. After both entities are connected and turned on, the optical instrument provides an acknowledgement that it is ready to collect data. The position of the probe is then determined and monitored by the automated surgical system and, when the tip of the probe is within the volume of operation, the system sends a “start” signal to the optical instrument to start data acquisition.

During operation, the optical instrument may send collected optical parameters and/or their derivation to the automated surgical system for storage, display, and/or other purposes. For example, the data (or information derived from the data) may be forwarded to a surgical microscope employed by a surgeon and/or displayed on one or more monitors that are part of the system. The automated surgical system can also send position information to the optical instrument, where it may be stored in combination with the optical data. When the tip of the optical probe exits the volume of operation, the automated surgical system sends a “stop” signal to the optical instrument to stop data collection and data transfer.

This protocol can be further refined to only pause or suspend the collection of optical data if the probe tip exits the volume of operation, and to resume collection if the probe tip reenters the volume of operation within a certain time threshold, or stop the acquisition altogether if the probe's tip remains outside of the volume of operation for more than the predetermined time threshold. Status information reported by the probe and/or determined by the automated surgical system can include position and/or angle of the probe, an indication as to whether the probe is collecting data, whether data collection has suspended or stopped. timestamps indicating when the probe started and/or stopped operating, and/or other information.

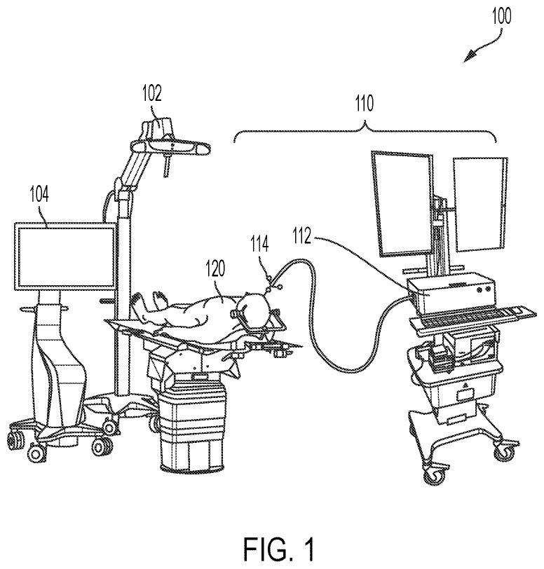

FIG. 1 is a block diagram of an automated surgical system with which an optical instrument (or other medical instrument) is integrated for automatic activation and deactivation, according to some embodiments.

In these embodiments, automated surgical system 100 includes sensor subsystem 102 and display subsystem 104 for displaying images captured or generated by sensor subsystem 102 and/or other instruments, such as optical instrument system 110, and for displaying preoperative images (e.g., a tomographic volume) for reference by the surgical team. Sensor subsystem 102 and/or display subsystem 104 may be mobile or stationary.

Optical instrument system 110 comprises console 112 and probe 114. Probe 114 is coupled to console 112, and console 112 is coupled to automated surgical system 100, via any suitable physical (e.g., wired, optical) or wireless communication mechanism(s). The console forwards data collected by probe 114 and/or information derived from the data (e.g., tissue characterizations) to the automated surgical system, where it may be displayed on a monitor, stored, forwarded to other equipment (e.g., a surgical microscope), etc. The console may also, or instead, communicate directly with other equipment. In addition, console 112 receives instructions from automated surgical system 100, including when to start and stop operation of probe 114.

Because automated surgical system 100 is programmed and calibrated to identify and determine the location of probe 114 whenever it can be viewed or sensed by sensor subsystem 102, monitors deployed as part of display subsystem 104 and/or console 112 of optical instrument 110 can show the precise location of the probe (and/or an active element of the probe, such as its tip) with relation to subject 120 of a current medical or surgical operation, as well as data collected by the probe and/or generated by the console (e.g., tissue measurements, intensity (e.g., of a fluorescent dye)). In some implementations (e.g., for neurosurgery), optical instrument 110 comprises a FLIm (Fluorescence Lifetime Imaging) device that characterizes tissue based on fluorescent emissions. In these implementations, a probe portion of optical instrument 110 transmits excitation light and collects fluorescence emissions, and console 112 performs associated tissue characterization (e.g., spectral separation and measurement).

Sensor subsystem 102 may include one or more cameras, infrared (IR) sensors, motion detectors, and/or other sensors. Therefore, in different embodiments, automated surgical system 100 may track the status (e.g., location, orientation) of probe 114 and/or other medical instruments visually, with infrared signals, or other non-visual electromagnetic radiation. Thus, a sensor within sensor subsystem 102 may receive infrared, radiofrequency, or other wireless signals emitted by the probe or by one or more emitters attached to the probe, or may emit an infrared (or other wireless) signal and receive a reflection of the signal from the probe or from one or more reflectors attached to the probe.

As one alternative, multiple cameras may be employed to track the probe, determine its location within the three-dimensional space encompassed by the cameras, and calculate its distance from other entities (e.g., a surgical patient, a defined volume of operation, members of the surgical team). U.S. Pat. No. 11,062,465 describes mechanisms and methods for tracking spatial positions of medical instruments, and is incorporated herein by reference.

Although not shown in FIG. 1, other equipment may be coupled to automated surgical system 100 and receive data collected or produced by optical instrument 110. For example, a surgeon may employ a surgical microscope for clear and detailed viewing of the surgical field, and a view through the microscope may be enhanced to show data produced by the instrument. For example, when the probe is used to measure or characterize tissue viewed through the microscope, that data may be overlaid upon the surgeon's view.

In some implementations, the optical instrument measures the fluorescence signals emanating from endogenous fluorophores naturally present in the patient's tissues and/or exogenous fluorophores administered to the patient prior to or during a procedure. Also, however, the optical instrument (e.g., console 112) may perform time-resolved measurements of the fluorescence signals. In other implementations, probe 114 may acquire an optical coherence tomography signal, an interferometric near-infrared spectroscopy signal, and/or a diffuse reflectance spectroscopy signal from the patient's tissue.

In these and/or other implementations, an instrument integrated with automated surgical system 100 may monitor or measure the estimated radiation exposure of tissue being operated upon and automatically pause operation of the instrument if the estimated radiation exposure exceeds a threshold, regardless of the location of the probe (e.g., within or without the volume of operation). Operation of the instrument may automatically resume when another estimation of the radiation exposure indicates that it is safe to do so, either because the probe has been moved to an area of tissue that has not reached its maximum exposure or because the operation has paused and the tissue has had time to recover. Similarly, to avoid photobleaching of exogenous molecular probes, radiation exposure may be monitored and irradiation may be curtailed for this purpose in addition to or instead of for the purpose of avoiding tissue damage.

In other embodiments, optical instrument system 110 is replaced or augmented with an instrument or tool having different functionality. For example, a tool integrated with automated surgical system 100 may instead (or also) perform tissue biopsy, conduct laser-induced thermal therapy, or deliver a drug or other substance (e.g., a dye, a radiological emitter).

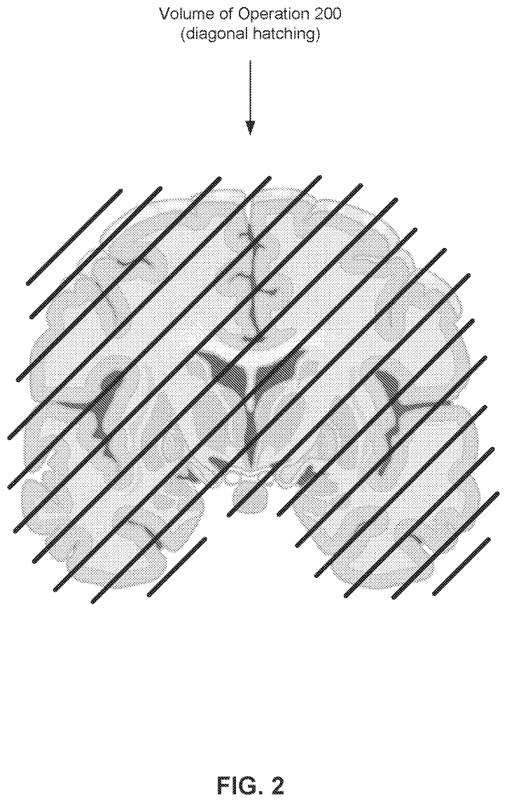

FIG. 2 depicts a volume of operation manually or automatically delineated for a relatively large area or volume, such as a subject's skull or brain (e.g., for brain surgery), according to some embodiments. In these embodiments, the automated surgical system or some other component analyzes preoperative medical (e.g., MRI, PET, CT) images, such as a three-dimensional tomographic volume, to detect the boundary of the surgical field (i.e., the skull or brain, the boundary of which contrasts with a background or surrounding color) and adds a buffer distance around that boundary to yield volume of operation 200, which is depicted with diagonal hatching. The boundary may then be matched to a subject of the surgery through calibration by, for example, moving the probe (e.g., a tip of the probe) to specific locations so that sensors (e.g., a camera subsystem) of the automated surgical system can match certain landmarks of the subject's body (e.g., base or tip of the subject's nose, temples, an earlobe) to the preoperative image.

Multiple images (e.g., in different planes or from different angles) may be analyzed to produce a three-dimensional result and provide medical personnel with full visibility of the volume of operation. Further, preoperative images may be integrated with a surgical microscope employed during the surgery, as well as with the optical instrument. This allows a surgeon to view, in real-time, not only the immediate area in which he/she is operating (i.e., via the microscope), but also the entire volume of operation (i.e., via the images), augmented with tissue data gathered by the probe, the location of which is also displayed.

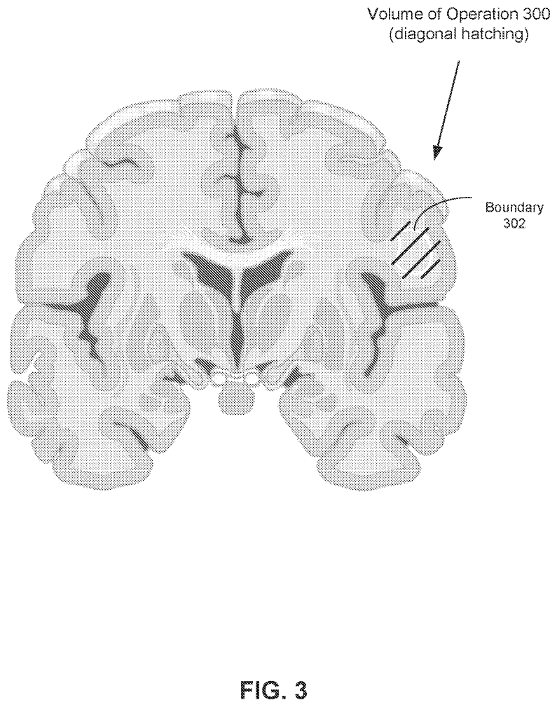

FIG. 3 depicts a volume of operation based on manual definition or tracing of the tissue being operated upon, according to some embodiments. In FIG. 3, boundary 302 (i.e., the semicircular shape) is drawn to demarcate tumorous tissue from surrounding tissue, and volume of operation 300 (marked with diagonal hatching) can then be determined accordingly. Again, the volume of operation and/or the boundary of the tumor (or other volume of interest) may be simultaneously drawn or rendered in multiple planes on multiple images to yield a three-dimensional volume. In some embodiments, as tissue is resected, the volume of operation may be automatically adjusted by the automated surgical system.

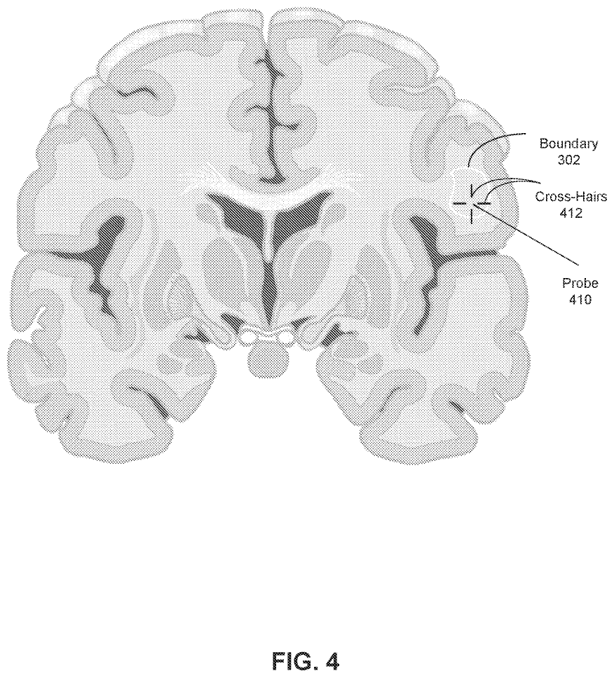

FIG. 4 illustrates the location of a handheld optical instrument probe 410, overlaid upon a medical image (e.g., a preoperative MRI image), and may be augmented with information derived from data collected by the probe, according to some embodiments. Cross-hatching denoting the volume of operation is omitted in the interest of clarity.

In these embodiments, a point of the probe is located at the intersection of horizontal and vertical cross-hairs 412. The diagonal line extending from the demarcated tumor tissue represents the probe, with the tip or operative point of the probe lying within the volume of operation. By referring to an image such as FIG. 4, again, usually displayed in multiple planes to yield a three-dimensional view, a surgeon can readily determine where the probe is located and, when the image is augmented with information derived from data collected by the probe, can also view pertinent characteristics of tissue near the probe. Similarly, the automated surgical system can automatically determine whether the probe (e.g., the tip of the probe) is within or without the volume of operation).

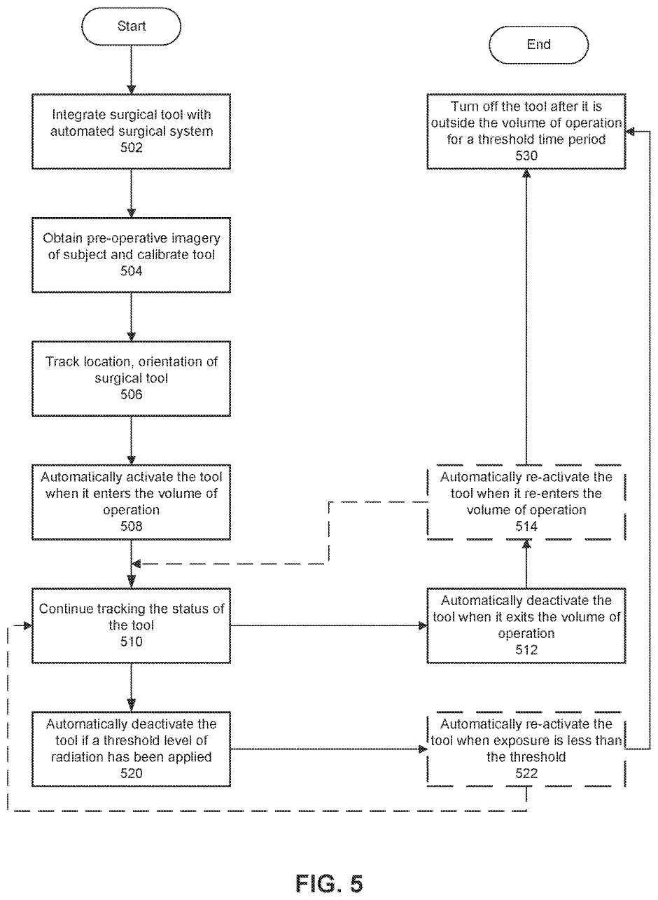

FIG. 5 is a flow chart demonstrating a method of automatically controlling operation of an instrument integrated with an automated surgical system, according to some embodiments. In other embodiments, one or more of the identified operations may be omitted, repeated, and/or performed in a different order. Accordingly, the specific arrangement of steps shown in FIG. 5 should not be construed as limiting the scope of any embodiment.

In operation 502, a medical/surgical tool for measuring, characterizing, examining, or otherwise assisting with a surgery or other medical procedure is integrated with an automated surgical system. The automated surgical system is capable of tracking and displaying this surgical tool and/or others, in any number of views on one or more display monitors.

As described above, in some embodiments, the tool is an optical instrument for characterizing tissue. During a medical procedure, data and/or images collected by a probe portion of this type of tool may be communicated to a console, which may be coupled to the automated surgical system, thereby making the collected data available to the entire medical team and other equipment, and also allowing it to be stored.

The tool may be configured with markings, visual patterns, signal emitters or reflectors, and/or other components to facilitate tracking of the tool by the automated surgical system. For example, reflective or visually distinctive tags, labels or beads may be affixed to the tool at one or more locations so that cameras and/or other sensors of the system are able to locate the tool and/or determine its orientation within three-dimensional space and with high accuracy and resolution (e.g., to the millimeter).

In operation 504, one or more images of a subject of a surgical operation are obtained via PET, CT, MRI, or other imaging system, and the tool is calibrated for the current medical procedure. For example, a tomographic volume may be obtained to show the entire volume of operation in three dimensions. Alternatively, multiple images in multiple planes or from different views may be correlated such that a given point in one image can be readily located or identified in another image that encompasses the same point.

Then the tool is calibrated with the automated surgical system and a subject of the current medical procedure. This may involve placing the tool close to (or in contact with) various points on the subject's body close to or within the volume of operation. Following calibration, while the tool is within a field of view or range of the automated surgical system's sensors, the location, orientation, and/or other status of the tool (e.g., online, offline, operative, inoperative) can be displayed via the automated surgical system and/or other display monitors. Similarly, data collected by the tool may also be displayed, such as tissue measurements and/or characterizations, temperature, radiation exposure, and so on.

In operation 506, after calibration is complete and the medical procedure is underway, the automated surgical system tracks the location and orientation of the surgical tool and may display its status as desired. In some implementations, the tool is wielded by a surgeon. As the tool approaches the subject of the procedure, the system continually or repeatedly determines whether the tool (or an operative portion of the tool, such as a tip) is within the defined volume of operation for the procedure. The tool may be kept in an off, offline, or standby mode of operation while outside the volume.

In operation 508, the tool (e.g., a tip of the tool) enters the defined volume of operation. Therefore, the automated surgical system commands the tool (or a console that couples the tool to the system) to turn on, go online, or otherwise become active. It should be noted that neither the wielder of the tool nor any other member of the surgical team need take action in order for the tool to be activated (other than move it within volume of operation). While within the volume of operation, the tool functions as designed to characterize or assess tissue, illuminate or irradiate tissue, capture images or video, apply a specified therapy, etc.

In operation 510, while the tool is active, the automated surgical system continues to monitor its status (location, orientation) and may also monitor data reported by the tool and/or other equipment. For example, if the tool (or some other instrument) emits radiation that may be harmful to the patient's tissue, the system may monitor the intensity and time of exposure. This information may be used, as described below, to avoid imparting a damaging level of radiation.

In operation 512, the tool (e.g., the tip of the tool) exits the volume of operation. In the currently described embodiments, the tool automatically transitions to a standby mode wherein it ceases normal operation but is capable of immediate (or nearly immediate) resumption of operation if needed. Therefore, although the tool ceases imaging, collecting data, characterizing tissue, etc., it can quickly resume operation if necessary.

In optional operation 514, the tool re-enters the volume of operation and is automatically reactivated and resumes normal operation. Specifically, without manual intervention by any person, the tool automatically resumes operation due to its reintroduction into the volume of operation. In some implementations, and in the interest of safety, the tool must reenter the volume within a specified period of time after its departure (e.g., one minute, five minutes) in order for operation to resume automatically. If optional operation 514 occurs, the method returns to operation 510 to resume monitoring the status of the tool.

In operation 520, the automated surgical system or the tool determines that a threshold or upper limit of radiation has been applied during a threshold period of time. This determination is enabled by tracking, throughout the procedure, the tissue areas that have been exposed and the duration of their exposure. To avoid damaging the patient, the tool and/or whatever other instrument is applying the radiation are automatically deactivated. The radiation exposure may continue to be monitored while the tool is deactivated.

In optional operation 522, sufficient time has passed that the tissue is deemed safe from damage from additional radiation. Therefore, the tool may be automatically reactivated if it is still needed and it remains within the volume of operation. If the tool is reactivated in this manner, the method returns to operation 510 to continue monitoring of the tool's status.

In operation 530, after the tool is located outside the volume of operation for a predefined period of time, or is removed from a field of view or sensing of the automated surgical system, it is turned off or placed offline. Whereas normal operation of the tool may have ceased or it may have been placed in a standby status immediately upon exiting the volume of operation, now it is powered off or otherwise made completely inactive. The method ends after operation 530.

As described above, various medical instruments or tools may be deployed for automatic activation and deactivation based on their proximity to a volume of operation, their orientation, measured or estimated levels of radiation applied to tissue within the volume of operation, and/or other factors. An optical instrument can be deployed to help medical personnel evaluate a patient's tissue to determine whether continued activity is required.

In some implementations, for example, an optical instrument can multi-spectrally and simultaneously evaluate excited state lifetimes of multiple fluorescent biomolecules within tissue (e.g., structural proteins, enzyme co-factors, lipid constituents, porphyrins). Fluorescence decay dynamics collected by the instrument yield information about molecular changes within the tissue, and lifetime measurements are independent of factors affecting the collected signal intensity (e.g., the presence of endogenous absorbers (such as blood) with the tissue and the surgical field, changes in excitation-collection geometry during in-vivo measurement). This allows the instrument to perform an ‘optical biopsy’ of the tissue and provide real-time tissue characterization and feedback to a surgeon, thereby enhancing the accuracy of treatment and resection and providing an extra margin of safety for the patient. In particular, prior to resection of tissue, it can be easily and quickly evaluated by the optical biopsy instrument to reaffirm or contradict the need for resection.

Injury to some tissue, such as brain tissue, may either be indicative of recurrence of a medical condition (e.g., cancer) or reflect a side effect of aggressive (e.g., radiation) treatment, and treatment for each cause may greatly differ. Therefore, during a surgical procedure it may be important to rapidly determine the cause or nature of damaged tissue without causing yet further damage. Use of a suitably configured optical instrument can be of great assistance, particularly when employed as part of a time-resolved fluorescence spectroscopy (TRFS) procedure.

An environment in which one or more embodiments described above are executed may incorporate a general-purpose computer or a special-purpose device such as a hand-held computer or communication device. Some details of such devices (e.g., processor, memory, data storage, display) may be omitted for the sake of clarity. A component such as a processor or memory to which one or more tasks or functions are attributed may be a general component temporarily configured to perform the specified task or function, or may be a specific component manufactured to perform the task or function. The term “processor” as used herein refers to one or more electronic circuits, devices, chips, processing cores and/or other components configured to process data and/or computer program code.

Data structures and program code described in this detailed description are typically stored on a non-transitory computer-readable storage medium, which may be any device or medium that can store code and/or data for use by a computer system. Non-transitory computer-readable storage media include, but are not limited to, volatile memory; non-volatile memory; electrical, magnetic, and optical storage devices such as disk drives, magnetic tape, CDs (compact discs) and DVDs (digital versatile discs or digital video discs), solid-state drives, and/or other non-transitory computer-readable media now known or later developed.

Methods and processes described in the detailed description can be embodied as code and/or data, which may be stored in a non-transitory computer-readable storage medium as described above. When a processor or computer system reads and executes the code and manipulates the data stored on the medium, the processor or computer system performs the methods and processes embodied as code and data structures and stored within the medium.

Furthermore, the methods and processes may be programmed into hardware modules such as, but not limited to, application-specific integrated circuit (ASIC) chips, field-programmable gate arrays (FPGAs), and other programmable-logic devices now known or hereafter developed. When such a hardware module is activated, it performs the methods and processes included within the module.

The foregoing embodiments have been presented for purposes of illustration and description only. They are not intended to be exhaustive or to limit this disclosure to the forms disclosed. Accordingly, many modifications and variations will be apparent to practitioners skilled in the art. The scope is defined by the appended claims, not the preceding disclosure.

What is claimed is:

1. A method of controlling a medical instrument for examining tissue during a medical procedure, the method comprising:
identifying a volume of operation in which the medical procedure will be performed;
repeatedly tracking a status of the instrument during the medical procedure;
automatically initiating operation of the instrument based on a first location of the instrument; and
automatically ceasing operation of the instrument based on a second location of the instrument.

2. The method of claim 1, further comprising:
coupling the instrument to an automated surgical system;
wherein the automated surgical system performs said repeated tracking of a status of the instrument with one or more sensors.

3. The method of claim 2, wherein the status of the instrument comprises a location and/or an orientation of the instrument.

4. The method of claim 2, wherein the status of the instrument comprises a measure of radiation to which the tissue has been exposed during the medical procedure.

5. The method of claim 2, further comprising:
communicating data to the automated surgical system from the instrument.

6. The method of claim 5, further comprising:
at the automated surgical system, displaying the data.

7. The method of claim 5, further comprising storing the data.

8. The method of claim 5, further comprising forwarding the data to a surgical microscope.

9. The method of claim 1, further comprising:
measuring, with the instrument, fluorescence signals from endogenous and/or exogenous fluorophores.

10. The method of claim 9, further comprising:
performing spectrally-resolved and/or time-resolved measurements of the fluorescence signals.

11. The method of claim 1, further comprising acquiring, with the optical instrument, one or more of:
a diffuse optical spectroscopy signal;
an optical coherence tomography (OCT) signal; and
an interferometric near-infrared spectroscopy signal.

12. The method of claim 1, wherein:
the first location is inside the volume of operation; and
the second location is outside the volume of operation.

13. The method of claim 1, further comprising:
pausing operation of the instrument based on a first estimated radiation exposure of tissue associated with the medical procedure; and
resuming the paused operation based on a second estimated radiation exposure of the tissue.

14. The method of claim 1, further comprising:
adjusting the volume of operation in response to removal of tissue during the surgery.

15. A system for controlling a medical instrument for examining tissue during a medical procedure, the system comprising:
an automated surgical system;
a medical instrument coupled to the automated surgical system and comprising a probe;
a communication link between the automated surgical system and the optical instrument; and
means for automatically activating and deactivating the medical instrument.

16. The system of claim 15, further comprising:
a video display that displays information derived from data collected by the probe.

17. The system of claim 16, wherein the video display further displays a position of the probe.

18. The system of claim 17, wherein:
the display of the position of the probe is overlaid upon an existing medical image; and
the image is obtained via one of:
magnetic resonance imaging (MRI);
positron emission tomography (PET); and
computed tomography (CT).

19. The system of claim 15, further comprising:
an array of infrared-reflecting reflectors coupled to the probe;
wherein the automated surgical system detects the infrared-reflecting reflectors to track a position and/or an orientation of the probe.

20. The system of claim 15, wherein the automated surgical system comprises:
at least one processor; and
memory storing instructions that, when executed by the at least one processor, cause the automated surgical system to:
track a location of a probe of the instrument during the surgery;
automatically initiate operation of the medical instrument based on a first location of the probe; and
automatically cease operation of the medical instrument based on a second location of the probe.

21. The system of claim 20, wherein the memory of the automated surgical system further stores instructions that, when executed by the at least one processor, cause the automated surgical system to:
automatically pause operation of the medical instrument when the probe departs a volume of operation in which the medical procedure is performed; and
automatically resume operation of the medical instrument when the probe reenters the volume of operation within a predetermined period of time after departing the volume of operation.

22. The system of claim 20, wherein the memory of the automated surgical system further stores instructions that, when executed by the at least one processor, cause the automated surgical system to:
forward to a surgical microscope data collected by the probe, for integration with a microscope display viewed by a surgeon.
